(19) United States
(12) Patent Application Publication
(10) Pub. No.: US 2005/0032743 A1
(43) Pub. Date: Feb. 10, 2005

(54) Compositions and method for steroid homeostasis

(76) Inventor: Dusan Miljkovic, San Diego, CA (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 10/648,975

(22) Filed: Aug. 26, 2003

Related U.S. Application Data
(60) Provisional application No. 60/406,427, filed on Aug. 27, 2002.

(51) Int. Cl. A61K 31/715; A61K 31/69; A61K 31/59

(52) U.S. Cl. 514/54; 514/64; 514/167

(57) ABSTRACT

Methods and compositions are directed to a dietary supplement comprising boron that is an a complex with a carbohydrate, wherein administration of the complex increases steroid levels, and particularly 25-hydroxy vitamin D3. Thus, contemplated compositions are useful for treatment of impaired steroid homeostasis, and especially beneficial for prevention and/or treatment of bone density loss. Particularly preferred compositions further include calcium, magnesium, and/or vitamin D in a nutritionally acceptable form.

This application claims priority to U.S. provisional patent application with the Ser. No. 60/406,427, which was filed Aug. 27, 2002, and which is incorporated by reference herein.

### FIELD OF THE INVENTION

The field of the invention is homeostasis of steroids in a biological system, especially as it relates to prophylaxis and maintenance of bones and teeth, and/or intervention in mineral loss in bones and teeth.

### BACKGROUND OF THE INVENTION

It is generally recognized that mineralization and demineralization of bones and teeth is closely regulated by parathyroid hormone and various steroids, primarily vitamin D-3, but also various estrogens (e.g., estradiol), dehydroepiandrosterone (DHEA), and testosterone. Depending on the particular nature of the steroid, mineralization and demineralization of bones and teeth is controlled at different levels.

For example, vitamin D-3 is thought to increase Ca²⁺ absorption from the gastrointestinal tract as well as incorporation of Ca²⁺ into the bone, and to decrease urinary excretion of Ca²⁺. Notably, vitamin D-3 is converted in the liver to a hydroxylated form, which is further hydroxylated in the kidneys to the most potent form calcitrol (1,25-dihydroxycholecalciferol). In another example, estrogens, and to a lesser extent testosterone, have been demonstrated to influence (and typically inhibit) bone demineralization. Interestingly, vitamin D-3 and DHEA have been implicated in the synthesis of estrogens, suggesting an intricate interplay between bone and teeth mineralization and various steroids.

Steroids typically comprise a hydrogenated cyclopentanoperhydrophenanthrene ring system and may be classified, depending on the degree of (de)saturation and/or the type of substituents into various groups, including progesterones, adrenocortical hormones, gonadal hormones, vitamin D, bile acids, sterols (e.g., cholesterol), saponins, etc. Especially where steroids have a regulatory function in a biological system, the amount of such steroids is typically tightly regulated. Typically, all or almost all of the regulatory systems for steroids comprise an up-regulatory component (e.g., transcriptional activation) and a down-regulatory component (e.g., site-specific hydroxylation and secretion).

Unfortunately, while inactivation/degradation pathways for steroids frequently remain functional over the entire life span of an organism, up-regulatory components and/or bioavailability of steroids typically begin to deteriorate (or are even shut down) in the second half of the life span. Consequently, many organisms begin to develop a steroid deficiency with increasing age. For example, estrogen levels drop dramatically in post-menopausal women, and occult vitamin D3 deficiency frequently develops in elderly individuals. It is generally known to provide individuals with steroid deficiency with synthetic hormones (e.g., in form of hormone replacement therapy) or vitamins (e.g., Vitamin D3). However, administration of steroids is frequently accompanied by undesirable side-effects. Moreover, administration of steroids only temporarily alleviates the effective concentration drop, but typically fails to maintain a desirable steady-state concentration.

Thus, although various methods are known to improve reduced steroid concentrations in an individual, all or almost all of them suffer from one or more disadvantages. Therefore, there is still a need to provide improved methods and compositions for steroid homeostasis.

### SUMMARY OF THE INVENTION

The present invention is directed to various aspects of compositions and methods of use for dietary supplements comprising a carbohydrate-boron complex. In one aspect of the inventive subject matter, a dietary supplement includes an isolated carbohydrate-boron (CHB) complex having a boron portion and at least one carbohydrate ligand complexed to the boron portion with a boron-ligand association constant of at least 50 (or higher), wherein the CHB complex is present in the dietary supplement in an amount sufficient to increase the steroid concentration in human plasma. Especially contemplated supplements further include in a nutritionally acceptable form calcium, magnesium, and/or vitamin D.

In further preferred aspects, the carbohydrate ligand is fructose, mannose, mannitol, sorbose, or sorbitol, and particularly contemplated CHB complexes include calcium fructoborate. Contemplated dietary supplements were demonstrated to elicit an increase of at least 10% (and more typically at least 20%) in 25-hydroxy-vitamin D3 and/or testosterone, thereby stimulating an increase in bone density.

Consequently, the inventors contemplate a method of increasing steroid concentration in a human in which an isolated carbohydrate-boron complex is provided, wherein the CHB complex has boron portion and at least one carbohydrate ligand complexed to the boron portion with a boron-ligand association constant of at least 50. In an other step, an instruction is provided to administer the CHB complex under a protocol that increases the steroid concentration in the human. Preferred administration protocols include those in which the CHB complex (optionally further comprising calcium, magnesium, and/or vitamin D) is orally daily administered at a dose of at least 1-10 mg over at least 30 days.

In a further contemplated aspect of the inventive subject matter, a method of marketing a dietary supplement has one step in which a dietary supplement selected from the group of a nutritionally acceptable form of boron, calcium, magnesium, and vitamin D is provided. In another step, printed information is provided that a combination of the nutritionally acceptable form of boron with at least one of the nutritionally acceptable form of calcium, magnesium, and vitamin D promotes bone health, wherein the nutritionally acceptable form of boron comprises an isolated carbohydrate-boron complex having a boron portion and at least one carbohydrate ligand complexed to the boron portion with a boron-ligand association constant of at least 50-250. It should be recognized that the printed information may further include information that the nutritionally acceptable form of boron increases a steroid concentration (e.g., testosterone and a 25-hydroxy vitamin D3) in the human.

Various objects, features, aspects and advantages of the present invention will become more apparent from the following detailed description of preferred embodiments of the invention.

### DETAILED DESCRIPTION

The inventors discovered that administration of selected boron-containing compounds modulates steroid homeostasis. More specifically, modulation of the steroid homeostasis results in an increased serum/plasma concentration of various steroids (particularly in their biologically active forms), which is believed to correlate with increased bone and teeth mineralization and/or decreased bone and teeth demineralization. Independently, the inventors also discovered that administration of selected boron-containing compounds significantly increases bone density, wherein such increase may be independently from the increased steroid concentration, or additively/synergistically.

The inventors still further observed that when vitamin D3 is administered together with a boron-containing compound, the concentration of the active form of vitamin D3 (25-hydroxylated) and serum half-life time is increased as compared to individuals who received a vitamin D3 dose without boron. Similarly, estrogen is thought to remain at a higher concentration and half-life time in an individual when relatively high concentration of boron are present in the individual.

Based on steric considerations of various carbohydrate-boron complexes and other boron-containing compounds, the inventors contemplate that various compounds with cis-vicinal diols generally have a configuration optimal for a cyclic borate formation. Moreover, the inventors contemplate that the positioning of the free hydroxyl groups in the partially hydrolyzed carbohydrate-complexed borate molecule is sufficiently similar to interact with the active site of hydroxylases that convert a mono-hydroxy compound into a cis-vicinal dihydroxy compound (particularly contemplated hydroxylases include hepatic P450-type enzymes, and especially those that convert monohydroxylated steroids into cis-vicinal dihydroxy steroids). Alternatively, or additionally, the partially hydrolyzed carbohydrate borate complex may be bound to a cis-dihydroxylated steroid and consequently slow down its further degradation. Since this first downstream degradation product also shows considerable vitamin D-3 activity, its slower degradation may increase vitamin D-3 activity.

Therefore, while not wishing to be bound to a particular theory or hypothesis, the inventors contemplate that the interaction between a boron-containing compound/carbohydrate-boron complex and a hydroxylase may be based on binding of the boron-containing compound/carbohydrate-boron complex (or its metabolites) as a transition state analog (for a conversion of the mono-hydroxy compound to a cis-vicinal dihydroxy compound), or may be based on non-covalent interactions (e.g., hydrogen bonds, hydrophilic interaction, ionic bond, etc.) between the boron-containing compound (or its metabolites) and functional groups in the active site. Consequently, the concentration of dihydroxylated steroids in systems with hydroxylases affected by a boron-containing compound/carbohydrate-boron complex is thought to decrease and consequently the rate of steroid degradation. Alternatively or additionally, since the dihydroxylated steroid also shows a considerable vitamin D-3 activity and since its degradation is slowed down, the overall vitamin D-3 effect is thought to be enhanced. It should be especially noted that this hypothesis is contrary to what has been described in U.S. Pat. No. 4,849,220, in which the inventors suggest that boric acid will increase the amount of hydroxylated steroids, a finding that the inventors could not reproduce in their set of experiments (see below).

Consequently, and viewed from one perspective, the inventors contemplate that contemplated carbohydrate-boron complexes (and their metabolites and degradation products) may be employed as an enzyme inhibitor for hydroxylases, and especially for hydroxylases that convert monohydroxylated steroids into cis-vicinal dihydroxy steroids. Since it is generally accepted that such hydroxylases provide a key step in the catabolism of steroid compounds, it should be appreciated that contemplated boron compounds may be employed as agents that increase a steady-state concentration of one or more steroids in a biological system (modulate steroid homeostasis) and/or increase the concentration of a steroid in the biological system. Alternatively or additionally, since the dihydroxylated steroid also shows a considerable vitamin D-3 activity and since its degradation is slowed down, the overall vitamin D-3 effect may be enhanced.

Thus, contemplated carbohydrate-boron complexes may be especially useful in treatment and/or prevention of disorders or conditions associated with a decreased concentration in one or more steroids. For example, where the steroid is an androgen, the inventors contemplate that administration of the compounds according to the inventive subject matter may help increase muscle mass and/or prevent loss of muscle mass. Similarly, where the steroid is a vitamin D or estrogen, the inventors contemplate that administration of the compounds according to the inventive subject matter may help increase bone density and/or prevent loss of density, especially when administered in combination with calcium, magnesium, and/or vitamin D.

With respect to the boron-containing compounds, it is especially contemplated that such compounds comprise boron or borate in a complexed form with at least one ligand, and particularly suitable compounds are described in U.S. Pat. Nos. 5,962,049, 5,985,842, and 6,080,425, all of which are incorporated by reference herein. The terms “boron-containing compound” and “carbohydrate-boron complex” as used herein expressly excludes boric acid and any salt thereof. Contemplated steroids include all known natural and synthetic steroids, and especially contemplated steroids include estrogens, testosterones, vitamin D, and their derivatives (e.g., prodrug forms, esters, salts, etc.). Preferred CHB complexes will have a boron portion and at least one carbohydrate ligand complexed to the boron portion with a boron-ligand association constant of at least 50, more typically at least 100, and even more typically at least 250.

Furthermore, where magnesium and/or calcium is administered in a nutritionally acceptable form, it is generally contemplated that all forms of magnesium and/or calcium are deemed suitable for use herein, and especially magnesium and calcium salts. The term “nutritionally acceptable” as used herein generally refers to all forms that are not acute toxic when administered at a dosage of 20 mg/kg, and especially include all forms of commercially available magnesium and calcium supplements (e.g., as salt, complexed, or as coral mineral). Similarly, where vitamin D is administered with contemplated carbohydrate-boron complexes, it should be recognized that all known vitamin D forms are deemed suitable for use herein (e.g., as isolated compound, or esterified to form a prodrug).

Administration of the boron-containing compound may follow any suitable protocol using any available route. However, it is generally preferred that the boron-containing compound/carbohydrate-boron complex is orally administered. Where oral administration is less preferred, alternative administrations especially include topical administration and injection.

### EXAMPLES

The following examples are provided for exemplary guidance to make and use the compounds and supplements according to the inventive subject matter. However, it should be recognized that numerous modifications may be made without departing from the inventive concept presented herein.

Synthesis of Contemplated Carbohydrate-Boron Complexes

When synthesizing boron compounds/complexes according to the present disclosure, one should generally follow accepted rules of chemical synthesis. Thus, if a ligand contains only one hetero-atom in its B-binding site, one takes four or more molar equivalents of it in respect to one molar equivalent of the starting boron compound. Further, if a ligand contains two or three hetero-atoms in its B-binding sites, one takes two or more molar equivalents of it in respect to one molar equivalent of the starting boron compound. Still further, if a ligand contains four or more hetero-atoms in its B-binding sites, one takes one or more equivalents of the ligand to one molar equivalent of the starting boron compound. Of course, the molar equivalent of the starting boron compound corresponds to its molecular formula if it contains one boron atom in it. If molecular formula contains more than one boron atom one divides molecular formula with a number of boron atoms containing in it. For example, if one starts with sodium tetraborate decahydrate, its molecular formula should be divided by four to obtain its molar equivalent.

### General Preparation Procedure Of Boron Compounds/Complexes

In most instances, the selected ligand at corresponding or slightly higher molar ratio than the boron compound, is mixed in suitable solvent (typically water) to form a relatively concentrated solution (e.g., between 10 wt % to 30 wt %). The reaction mixture is stirred until all solids are dissolved. Where boric acid is the boron compound, subsequent neutralization may be performed (e.g., NaHCO₃, KHCO₃, CaHCO₃, etc.). The so prepared complex in solution may then be used for final preparation of the supplement, or further purified.

### Example 1

### Calcium Boro-Mannitol (Calcium Mannitolo-Borate)

Boric acid (1.24 g; 20 mmoles) and mannitol (7.28 g; 40 mmoles) were dissolved in water (20 ml) at 60°-70° C. After cooling down to room temperature, solid calcium carbonate (1 g; 10 mmoles) was gradually added the solution. During the addition of calcium carbonate carbon dioxide evolved. When all calcium carbonate dissolved and carbon dioxide evolution ceased (about 30 minutes), ethanol (80 ml) was added. A viscous (semi-solid) heavy layer separated out, and the upper aqueous-ethanolic solution was decanted. A new portion of ethanol was added (80 ml), and the crystalline complex separated out upon stirring at room temperature. The crystalline complex was filtered, washed with ethanol (40 ml), and dried in a vacuum desiccator to yield pure crystalline Ca-mannito-borate (7 g; 90% of theoretical yield).

A similar procedure can be carried out using other cations, including magnesium and potassium. Furthermore, alternative carbohydrates may also be employed and particularly include fructose. In yet other alternative aspects, ascorbates, and particularly ascorbic acid may be used instead of carbohydrates. It should be recognized that the preparation of boron complexes with these alternative ligands will proceed following substantially the same protocol as outlined above.

### Example 2

### Sodium Serine/Borate Complex

Sodium tetraborate (0.804 g; 4 mmoles) and serine (3.2 g; 32 mmoles) were mixed in water (20 ml) at room temperature and stirred for 0.5 to 1 hour at room temperature. The final concentration of the components may then be adjusted to a desired level (e.g., 2-4 mg B/ml). The complex may then be used directly as a liquid, or crystallized from the solvent and further purified as desired.

Biological Effects of Contemplated CHB Complexes

### Example 3

### Effect of Calcium Fructoborate on Selected Steroids

A group of healthy volunteers was given after obtaining their informed consent a daily oral dose of 6 mg Calcium fructoborate (as prepared in Example 1 above) for a period of 60 days, and the serum/plasma concentrations of testosterone, 25-hydroxy vitamin D, and dehydroepiandrosterone was determined in an independent clinical laboratory. Table 1 below summarizes the results in which TES refers to ng/ml Testosterone, DHEA refers to mcg/ml dehydroepiandrosterone, and VITD refers to ng/ml Vitamin D. The first number is the serum/plasma concentration at the beginning of the trial while the second number is the serum/plasma concentration after 60 days.

TABLE 1

| VOLUNTEER | TES | DHEA | VITD |
|---|---|---|---|
| C.V. | 11.9/14.1 | 1.9/2.1 | 7.9/9.9 |
| S.J. | 6.3/8.2 | 1.9/2.1 | 12.1/16.1 |
| M.J. | 6.2/7.8 | 3.5/4.1 | 11.8/15.8 |
| M.L. | 6.2/8.0 | 2.75/2.95 | 9.3/12.0 |
| Average increase | 25% | 11% | 28% |

As can be clearly seen, administration of calcium fructoborate over the above specified time significantly increased the serum/plasma concentration of various steroids, which is expected to significantly influence mineralization/demineralization of bone and teeth in human receiving contemplated complexes as dietary supplement.

### Example 4

### Effect of Calcium Fructoborate on Bone Density

Vitamin D deficient Sprague Dawley rats (21 days old) were divided into three groups and fed a regulated vitamin D deficient diet (0.47% Ca, 0.3% P) over a period of 9 weeks. The diet of the first group was supplemented with calcium fructoborate at 30 mcg/gm, the diet of the second group was supplemented with calcium fructoborate at 185 mcg/gm, and the diet of the third group was left unchanged as a control group. At the end of the test period animals of all three groups were evaluated for increase in vitamin D levels and increase in bone density.

Interestingly, in this set of experiments using severely vitamin D deficient test animals, calcium fructoborate did not conserve or promote biosynthesis of vitamin D. This finding was further supported by the fact that the fructoborate failed to promote growth of the animals, or raised the serum calcium concentration above the control. However, when the three test groups were compared for bone ash increase, it became evident that fructoborate increased the bone density, even under vitamin D deficiency conditions. Specifically, the bone ash increase in rats of the first test group was 1.4% over the control, and the bone ash increase in rats of the second test group was 5.8% over the control.

Therefore, the inventors particularly contemplate a dietary supplement that includes an isolated carbohydrate-boron complex having a boron portion and at least one carbohydrate ligand complexed to the boron portion with a boron-ligand association constant of at least 250, wherein the carbohydrate-boron complex is present in the dietary supplement in an amount sufficient to increase a plasma steroid concentration of a human ingesting the dietary supplement. Particularly preferred supplements will further include at least one of a nutritionally acceptable form of calcium, magnesium, and vitamin D. As used herein, the term “isolated carbohydrate-boron complex” refers to all carbohydrate-boron complexes that are either synthetically prepared or prepared from a source in which such complexes naturally occur (e.g., various fruits). Viewed from another perspective, the term “isolated carbohydrate-boron complex” expressly excludes carbohydrate-boron complexes in an environment from which they have not been isolated/enriched (e.g., various fruits).

Particularly preferred amounts of contemplated complexes include those where the isolated carbohydrate-boron complex is present in at least 0.1 mg, more typically in at least 1.0 mg, and most preferably in between about 5.0-10.0 mg in the dietary supplement. However, suitable amounts may even be higher (e.g., between 10 mg-50 mg, and even more) where appropriate. Thus, one preferred daily administration may result in an oral uptake of contemplated CHB complexes of at least 1 mg, more typically at least 10 mg, and most typically at least 25 mg (e.g., over a period of at least 30 days, and more typically at least 60 days). For example, time release formulations, or formulations for populations with moderate to severe boron deficiency may benefit from higher amounts of isolated carbohydrate-boron complex in a supplement. On the other hand, amounts of less than 0.1 mg may also be suitable, especially where one or more steroids are administered to the person that receives the isolated carbohydrate-boron complex. Particularly preferred carbohydrate-boron complex include those in which the carbohydrate ligand is fructose, mannose, mannitol, sorbose, or sorbitol, and especially preferred complexes are calcium fructoborate (supra).

Similarly, the quantities of the nutritionally acceptable form of calcium, magnesium, and/or vitamin D may vary substantially. However, it is generally preferred that calcium is present in the supplement in an amount of between about 10 mg to about 1000 mg, and more preferably between about 100 mg and 500 mg. With respect to the quantities of magnesium, it is typically preferred to include magnesium in an amount of between about 5 mg-500 mg, and more preferably between about 50 mg and 250 mg. It should be recognized, however, that magnesium and/or calcium may be present in higher quantities if needed (e.g., in the course of treatment of moderate to severe osteoporosis). On the other hand, where the supplement is employed for maintenance of bone/teeth health, lower amounts for calcium and/or magnesium are also contemplated (e.g., 1-10 mg calcium, or 0.5-5 mg magnesium). Vitamin D may is preferably present in contemplated supplements in amounts of between about 100 I.U. to 1000 I.U., and most preferably between about 300 I.U. to 600 I.U. However, where desired, higher amounts may also be used (e.g., up to 5000 I.U., and in rare cases even higher). Likewise, lower concentrations (e.g., between about 10 I.U and 100 I.U.) may be employed where long-term administration is particularly preferred.

Of course it should be recognized that the carbohydrate-boron complex and the calcium, magnesium, and/or vitamin D in contemplated dietary supplements may be combined into a single dosage form (e.g., tablet, capsule, etc.) or provided in a separate manner. Combination of such ingredients will advantageously simplify administration, however, separate administration may allow for “customization” to the needs of a specific person ingesting such supplements.

The inventors further contemplate that dietary supplements according to the inventive subject matter as well as contemplated carbohydrate-boron complexes will increase the steroid concentration of various steroids (supra), however, particularly observed that 25-hydroxy-vitamin D3 was significantly increased upon administration over a period of at least 14 days, more typically at least 30 days, and most typically at least 60 days. The increase was shown to be at least 15%, more commonly at least 20%, and in some cases even more than 25%. Similarly, testosterone levels increased commonly at least 15%, more commonly at least 20%, and in some cases even more than 25% during administration over at least 14 days, more typically at least 30 days, and most typically at least 60 days. Such increase was thought to stimulate an increase in bone density.

Therefore, the inventors contemplate a method of increasing a steroid concentration in a human, in which in one step an isolated carbohydrate-boron complex is provided having a boron portion and at least one carbohydrate ligand complexed to the boron portion with a boron-ligand association constant of at least 250. In another step, an instruction is provided to administer the carbohydrate-boron complex under a protocol that increases the steroid concentration in the human. Based on the data presented above and other results (data not shown), the inventors generally contemplate that preferred administration protocols directs a person to daily oral administration of at least 1 mg of the carbohydrate-boron complex over at least 30 days, wherein in at least some cases co-administration of at least one of a nutritionally acceptable form of calcium, magnesium, and vitamin D is advised. Such methods are thought to increase the steroid level of testosterone, estrogen, and/or a 25-hydroxy vitamin D3, and with that will stimulate an increase in bone density in the person ingesting such supplements.

Consequently, a method of marketing will include one step in which at least one of a dietary supplement selected from the group of a nutritionally acceptable form of boron, calcium, magnesium, and vitamin D is provided. In another step, printed information is provided that a combination of the nutritionally acceptable form of boron with at least one of the nutritionally acceptable form of calcium, magnesium, and vitamin D promotes bone health, wherein the nutritionally acceptable form of boron preferably comprises an isolated carbohydrate-boron complex having a boron portion and at least one carbohydrate ligand complexed to the boron portion with a boron-ligand association constant of at least 250.

Such printed information may advantageously include a sales brochure or poster, a package insert, and/or label on the dietary supplement, which may further include information that the nutritionally acceptable form of boron increases a steroid concentration (e.g., estrogen, testosterone, or 25-hydroxy vitamin D3) in the human.

Thus, specific embodiments and applications of compositions and method for steroid homeostasis have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A dietary supplement comprising:
an isolated carbohydrate-boron complex having a boron portion and at least one carbohydrate ligand complexed to the boron portion with a boron-ligand association constant of at least 50;
wherein the carbohydrate-boron complex is present in the dietary supplement in an amount sufficient to increase a plasma steroid concentration of a human ingesting the dietary supplement; and
at least one of a nutritionally acceptable form of calcium, magnesium, and vitamin D.

2. The dietary supplement of claim 1 wherein the carbohydrate ligand is selected from the group consisting of fructose, mannose, mannitol, sorbose, and sorbitol.

3. The dietary supplement of claim 2 wherein the carbohydrate-boron complex is a calcium salt of fructoborate.

4. The dietary supplement of claim 3 comprising the nutritionally acceptable form of calcium.

5. The dietary supplement of claim 3 comprising the nutritionally acceptable form of magnesium.

6. The dietary supplement of claim 3 comprising the nutritionally acceptable form of Vitamin D.

7. The dietary supplement of claim 3 wherein the steroid is 25-hydroxy-vitamin D3, and wherein the increase is at least 10%.

8. The dietary supplement of claim 3 wherein the steroid is testosterone, and wherein the increase is at least 10%.

9. The dietary supplement of claim 3 wherein the increase of the steroid concentration stimulates an increase in bone density.

10. The dietary supplement of claim 1 wherein the carbohydrate-boron complex and the at least one of a nutritionally acceptable form of calcium, magnesium, and vitamin D are combined in a tablet or capsule.

11. The dietary supplement of claim 1 wherein the carbohydrate-boron complex is present at an amount sufficient to increase bone density.

12. A method of increasing a steroid concentration in a human, comprising:
providing an isolated carbohydrate-boron complex having a boron portion and at least one carbohydrate ligand complexed to the boron portion with a boron-ligand association constant of at least 50; and
providing an instruction to administer the carbohydrate-boron complex under a protocol that increases the steroid concentration in the human.

13. The method of claim 12 wherein the carbohydrate ligand is selected from the group consisting of fructose, mannose, mannitol, sorbose, and sorbitol.

14. The method of claim 13 wherein the carbohydrate-boron complex is a calcium salt of fructoborate.

15. The method of claim 12 wherein the protocol comprises daily oral administration of at least 1 mg of the carbohydrate-boron complex over at least 30 days.

16. The method of claim 12 further comprising a step of co-administering at least one of a nutritionally acceptable form of calcium, magnesium, and vitamin D.

17. The method of claim 12 wherein the steroid is at least one of a testosterone, an estrogen, and a 25-hydroxy vitamin D3, and wherein the increase in the steroid concentration stimulates an increase in bone density in the human.

18. A method of marketing a dietary supplement, comprising:
providing at least one of a dietary supplement selected from the group of a nutritionally acceptable form of boron, calcium, magnesium, and vitamin D;
providing printed information that a combination of the nutritionally acceptable form of boron with at least one of the nutritionally acceptable form of calcium, magnesium, and vitamin D promotes bone health; and
wherein the nutritionally acceptable form of boron comprises an isolated carbohydrate-boron complex having a boron portion and at least one carbohydrate ligand complexed to the boron portion with a boron-ligand association constant of at least 250.

19. The method of claim 18 wherein the printed information further includes information that the nutritionally acceptable form of boron increases a steroid concentration in the human.

20. The method of claim 19 wherein the steroid is at least one of a testosterone, an estrogen, and a 25-hydroxy vitamin D3.
